(19) United States
(12) Patent Application Publication
(10) Pub. No.: US 2024/0377413 A1
(43) Pub. Date: Nov. 14, 2024

(54) DUAL CAPTURE METHOD FOR ANALYSIS OF ANTIBODY-DRUG CONJUGATES

(71) Applicant: BRISTOL-MYERS SQUIBB COMPANY, Princeton, NJ (US)

(72) Inventors: Srikanth KOTAPATI, Fremont (IN); Gavin David DOLLINGER, San Francisco, CA (US); Arvind RAJPAL, San Francisco, CA (US)

(21) Appl. No.: 17/760,692

(22) PCT Filed: Sep. 15, 2020
(86) PCT No.: PCT/US2020/050798

§ 371 Date: Mar. 15, 2022

Related U.S. Application Data
(60) Provisional application No. 62/900,892, filed on Sep. 16, 2019.

(51) Int. Cl. G01N 33/68

(52) U.S. Cl. CPC G01N 33/6857; G01N 33/6848; G01N 2333/924; G01N 2333/96466; G01N 2333/98

(57) ABSTRACT

A method for analyzing whether, and to what extent, a transformation has occurred in the partner molecule component of an antibody-drug conjugate.

### CROSS-REFERENCE TO RELATED APPLICATIONS

This application claims the benefit under 35 U.S.C. § 119 (e) of U.S. Provisional Application Ser. No. 62/900,892, filed Sep. 16, 2019; the disclosure of which is incorporated herein by reference.

### BACKGROUND OF THE DISCLOSURE

This disclosure relates to the analysis of antibody-drug conjugates to determine if a transformation has occurred in the partner molecule attached thereto.

An antibody-drug conjugate (also referred to as an “ADC”, “conjugate,” or “immunoconjugate”) is a biologic that has attracted significant current interest. An ADC comprises three components: (1) an antibody, (2) a partner molecule, and (3) a linker covalently joining the first two components. The number of partner molecules attached to each antibody is referred to as the “drug-antibody ratio” or “DAR” and can vary, but commonly is from about 1 to about 8.

Frequently, the partner molecule (also referred to as the “drug,” “warhead,” or “payload”) is a therapeutic agent such as an anti-cancer drug. The antibody is one whose antigen is expressed by a target cell or tissue. The antibody, through its binding to the antigen, serves to deliver the ADC to the target. Once there, cleavage of the covalent link or degradation of the antibody results in the release of the drug at the target location. Conversely, while the conjugate is circulating in the blood system, the therapeutic agent is held in an inactive mode because of its covalent linkage to the antibody, thus reducing the risk of systemic side effects. For a review on ADCs in anti-cancer treatment, see Gerber et al. 2013.

ADCs that have received marketing approval treatment include inotuzumab ozogamicin (BENSPONSA™), fam-trastuzumab deruxtecan-nxki (ENHERTU™), enfortumab vedotin-ejfv (PADCEV™), polatuzumab vedotin-pliq (POLIVY™), sacituzumab govitecan (TROVELDY™), brentuximab vedotin (ADCETRIS™) and ado-trastuzumab emtansine (KADCYLA™).

It is important to have assays available for assessing the structural integrity of an ADC at various stages of development or production. One obvious matter to assay for is premature release of the drug, by determining the amount, if any, of free drug. A more difficult proposition is to assay for a transformation in the drug while still attached to the ADC, for example cleavage of an ester or amide bond on the drug, causing a structural change that negatively impacts the activity of the drug when it is eventually released from the ADC.

Kaur et al. 2013 and Darwish et al. 2017 disclose methods for analysis of an ADC where the partner molecule is attached to the light chain of the antibody.

However, frequently the partner molecule/drug is attached to the heavy chain of the antibody, for example at the Fc fragment thereof. Thus, an efficient method for analyzing for a transformation in the partner molecule while still attached to the heavy is desirable.

Full citations for the documents cited herein by first author or inventor and year are listed at the end of this specification.

### BRIEF SUMMARY OF THE DISCLOSURE

Mass spectrometry (“MS”) is a method of choice for analyzing a biologic sample for a transformation thereto. However, a key obstacle to measuring transformation of a partner molecule in an ADC by MS is the huge disparity in size between the partner molecule, typically having a molecular weight of several hundred daltons, and the antibody, at about 150,000 daltons (150 kDa), distributed between two heavy chains of 50 kDa each and two light chains of 25 kDa each, creating signal-to-noise issues.

This disclosure provides a dual capture method for analyzing whether, and to what extent, a transformation has occurred in a partner molecule while attached to the antibody heavy chain of an ADC. If the analysis is repeated over time, an estimate of the rate of transformation—i.e., the stable half-life of the partner molecule—can be estimated.

In one aspect, this disclosure provides a method for analyzing whether, in an antibody-drug conjugate (ADC) having a partner molecule attached to the Fc fragment of the antibody, a transformation has occurred in the partner molecule, comprising the steps of:
a. capturing the ADC on first beads having an affinity for the F(ab′)₂ region of the antibody;
b. digesting the ADC captured on the first beads in a digestion medium comprising a protease that cleaves the antibody into an F(ab′)₂ fragment and an Fc fragment having the partner molecule attached thereto and optionally a deglycosylating enzyme, whereby the F(ab′)₂ fragment remains attached to the first beads and the Fc fragment is released;
c. capturing the released Fc fragment having the partner molecule attached thereto on second beads having an affinity for the Fc fragment;
d. eluting with an acidic eluent the Fc fragment having the partner molecule attached thereto from the second beads;
   and
e. obtaining a mass spectrometric analysis of the eluted Fc fragment having the partner molecule attached thereto, to determine if a transformation occurred in the partner molecule.

In another aspect, there is provided a method for analyzing whether, in an antibody-drug conjugate (ADC) having a first partner molecule attached to the Fc fragment of the antibody and a second partner molecule attached to the Fd′ fragment of the antibody, a transformation has occurred in either the first or second partner molecule, comprising the steps of:
a. capturing the ADC on first beads having an affinity for the F(ab′)₂ region of the antibody;
b. digesting the ADC captured on the first beads in a digestion medium comprising a protease that cleaves the antibody into an F(ab′)₂ fragment having the second partner molecule attached thereto and an Fc fragment having the first partner molecule attached thereto and optionally a deglycosylating enzyme, whereby the F(ab′)₂ fragment remains attached to the first beads and the Fc fragment is released;
c. capturing the released Fc fragment having the first partner molecule attached thereto on second beads having an affinity for the Fc fragment;
d. eluting with an acidic eluent the Fc fragment having the first partner molecule attached thereto from the second beads;
e. eluting with an acidic solvent the F(ab′)₂ fragment having the second partner molecule attached thereto from the first beads;
f. reducing disulfide bonds in the eluted F(ab′)₂ to produce an Fd′ fragment having the second partner molecule attached thereto;
   and
e. obtaining a mass spectrometric analysis of the eluted Fc fragment containing the first partner molecule attached thereto and the Fd′ fragment having the second partner molecule attached thereto, to determine if a transformation occurred in the first and second partner molecules.

### BRIEF DESCRIPTION OF THE DRAWING(S)

FIG. 1 shows schematically a dual capture method for analyzing for a transformation in a partner molecule attached to the Fc fragment of the antibody in an ADC.

FIGS. 2A and 2B each show schematically a dual capture method for analyzing for transformations in an ADC having first and second partner molecules respectively attached to the Fc and F(ab′)₂ regions of the antibody. In the instance of FIG. 2B, the ADC additionally has a third partner molecule, which is attached to the light chain.

FIG. 3 shows schematically a comparative single capture method.

FIGS. 4A, 4B, 4C, and 4D show the results for transformation analysis over a 14-day period for an ADC-5 (as described hereinbelow) having a partner molecule attached to the Fc fragment thereof, using a dual capture method.

FIGS. 5A, 5B, 5C, and 5D show the results for transformation analysis for an ADC-6 (as described hereinbelow) having a first partner molecule attached to the Fc fragment thereof and a second partner molecule attached to the F(ab′)₂ region thereof, using a dual capture method. The results are for transformation analysis of the first partner molecule.

FIGS. 6A, 6B, 6C, and 6D show the results for transformation analysis for ADC-6,using a dual capture method, for transformation of the second partner molecule.

FIGS. 7A, 7B, 7C, and 7D show the results for transformation analysis period for an ADC-3 (as described hereinbelow) having a partner molecule attached to the Fc fragment thereof, using a single capture method.

FIGS. 8A, 8B, and 8C show data for the incomplete digestion of ADC-3 when processed according to the single capture scheme of FIG. 3.

FIGS. 9A, 9B, and 9C show data for the incomplete digestion of ADC-5 when processed according to the single capture scheme of FIG. 3.

FIGS. 10A, 10B, 10C, 10D, and 10E show how the digestion of ADC-5 is complete if a dual capture according to FIG. 1 or FIG. 2A is used.

FIGS. 11A, 11B, 11C, and 11D show the results for transformation analysis of the light chain of an ADC-7 (as described hereinbelow), which has a partner molecule attached to it light chain, Fd′ fragment, and Fc fragment. FIGS. 12A, 12B, 12C, and 12D show the results for transformation analysis of the Fd′ fragment of ADC 7. FIGS. 13A, 13B, 13C, and 13D show the results for transformation analysis of the Fc fragment ADC-7.

### DETAILED DESCRIPTION THE DISCLOSURE

### Embodiments

Transformations to the partner molecule that that can be assayed according to this disclosure preferably are those that cause a change in its mass, making detection by mass spectrometry feasible. Examples of such changes include, but are not limited to, hydrolysis of an ester group, hydrolysis of an amide group, ring opening of a lactone or lactam, oxidation of an alkyl moiety, and oxidation of an alcohol group. The transformation can be one mediated by an enzyme or one that occurs due to an ambient chemical condition such as pH or oxygen content. The present disclosure provides a method of breaking apart an ADC into smaller fragments of defined size that are amenable to analysis by liquid chromatography/high resolution mass spectrometry, thus overcoming the signal-to-noise issue attendant to analyzing an intact ADC, with its huge size disparity between a full length antibody and a partner molecule.

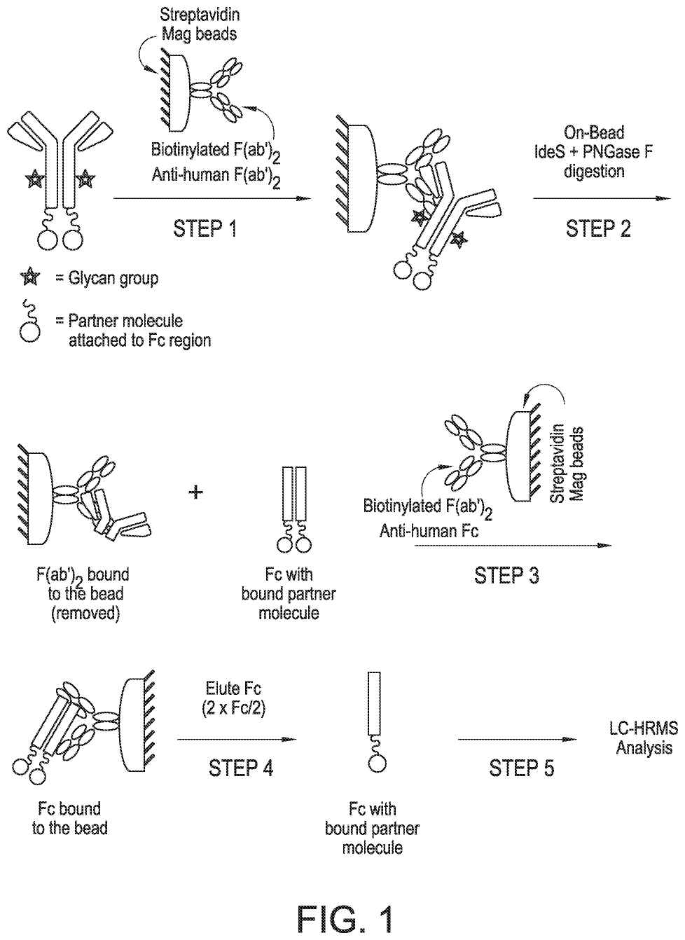

An embodiment is illustrated by FIG. 1. As an exemplar, the ADC depicted in the figure is an ADC of a glycosylated (★) human antibody having two drug moieties (○) attached thereto, one each in the Fc fragment.

In STEP 1 the ADC is captured on streptavidin-coated beads onto which is bound a biotinylated F(ab′)₂ fragment having specificity for human F(ab′)₂. The anti-human F(ab′)₂ affinity of the biotinylated fragment results in capture of the ADC onto the beads. Preferably, the beads are magnetic, for ease of handling and separation. The “polarity” of the beads can be reversed, in the sense that, instead, one can use biotin coated beads to which are bonded the streptavidin-labeled F(ab′)₂ fragment.

In STEP 2 the captured ADC is digested with a protease such as immunoglobulin degrading enzyme from Streptococcus pyogenes (“IdeS”) and a deglycosylating enzyme such as peptide: N-glycosidase F (“PNGase F”). The protease cleaves the antibody of the ADC into an F(ab′)₂ fragment, which remains bound to the beads, and an Fc fragment, which is released into solution. The PNGase F removes the glycan from the antibody. If the antibody is not glycosylated, PNGase F digestion is not needed and this enzyme can be omitted from the digestion medium. Preferably, the digestion pH is kept approximately neutral. (In theory, pepsin, which will also cleave an antibody to yield Fc and F(ab′)₂ fragments could be used, but is less desirable because pepsin requires low pH for activity.) The following digestion conditions are representative: 100 units each of IdeS and PNGase F (if used), 1.5 h incubation at 37° C., HBS-EP (pH7.4) buffer. A surfactant in the buffer helps keep the beads well mixed, but is not essential. Alternatively to IdeS, a different protease such as IdgE (available from Genovis Inc., Cambridge, MA 02142, as FabALACTICA™) or IdeZ (a protease derived from Streptococcus equi subspecies zooepidemicus, available from Promega Corporation, Madison, WI 53711) can be used. Alternatively to PNGase F, another deglycosylating enzyme, such as EndoS or EndoS2 (both endoglycosidases from Streptococcus pyogenes), can be used. Preferably, the protease cleaves the antibody at a position from 10 amino acids above to 10 amino acids below the hinge disulfides, more preferably at a position from 5 amino acids above to 5 amino acids below the hinge disulfides. The Fc fragment is dimeric, held together by non-covalent interactions and retains attached thereto the drug molecule. (If the cleavage position is below the hinge disulfides, they will also be present in the Fc fragment will contribute to holding it together as a dimer.) Preferably, the cleavage position is below the hinge disulfides, so that they are located in the F(ab′)₂ fragment. IdeS and IdeZ both cleave at the same position, namely below the hinge disulfides.

In STEP 3, streptavidin beads having anti-human Fc biotinylated F(ab′)₂ bound thereto is used to capture the Fc fragment released in STEP 2. This second affinnity capture is critical for selectively capturing the Fc fragment from the digestion mixture containing surfactant and enzymes (IdeS and optionally PNGase F). As in the case of the first capture of STEP 1, the beads are preferably magnetic and the streptavidin-biotin “polarity” can be reversed.

In STEP 4, the captured Fc fragment is eluted from the beads with an acidic eluent, to provide monomeric (disassociated) Fc fragments having the drug molecule attached thereto. Different elution solvents, including (a) 1% formic acid, (b) acetic acid, (c) 0.1 M glycine hydrochloride, and 12 mM hydrochloric acid can be used. A preferred eluent is 1% formic acid. The addition of a water miscible organic solvent (e.g., acetonitrile, methanol, or isopropanol) can improve Fc fragment recovery. Best results were obtained with about 1% formic acid in about 25% acetonitrile, volume/volume. With a higher concentration of acetonitrile, such as 30%, the Fc fragments could crash out of solution. (If, in STEP 2, the protease cleaves the antibody above the hinge disulfides so that they are located in the Fc fragment, they can be reduced with TCEP or other suitable reducing agent as discussed below.)

In STEP 5, liquid chromatography-high resolution mass spectrometry (LC-HRMS) is used to analyze for the occurrence (or not) of a transformation. Alternatively, capillary electrophoresis coupled with high resolution mass spectrometry (CE-HRMS) can be used.

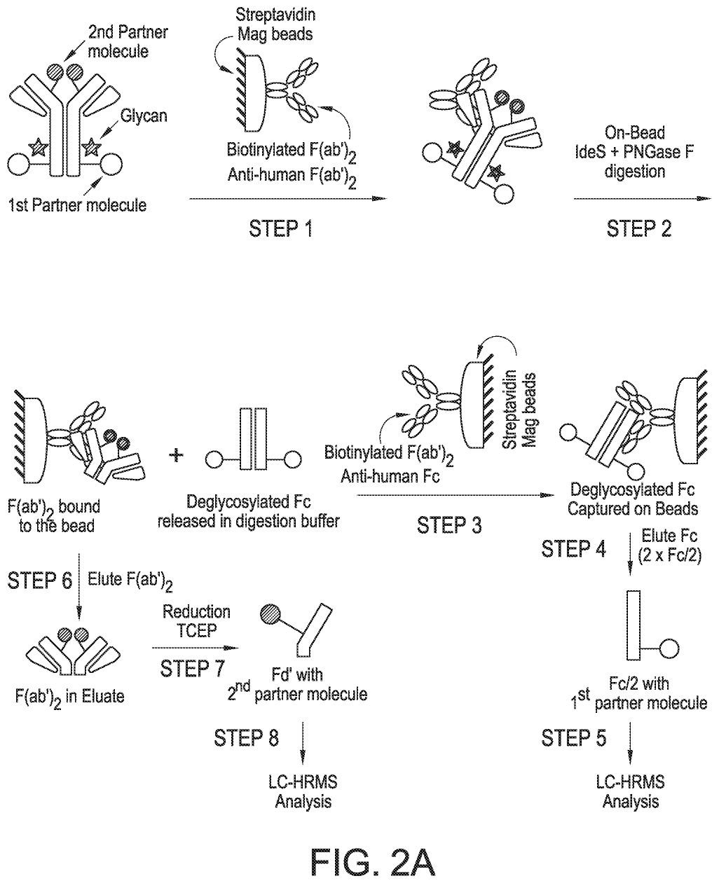

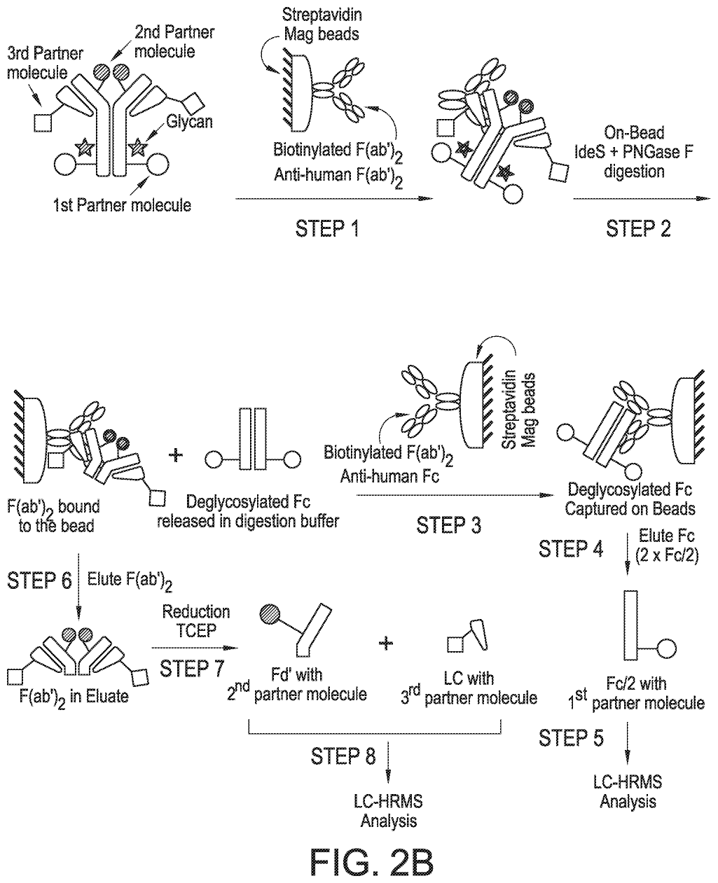

Reference is now made to FIG. 2A, which shows how the dual capture method disclosed herein can be used to analyze a double ADC having a first partner molecule (●) attached to the Fc fragment of a glycosylated (★) antibody and a second partner molecule (●) attached to its F(ab′)₂ region, specifically the Fd′ fragment. STEPS 1-5 are as described in respect of FIG. 1. Additionally, in STEP 6 the bead-bound F(ab′)₂ fragment is eluted with an acidic eluent, in the same manner as STEP 4 of this figure and of FIG. 1. In STEP 7, reduction of the disulfide bonds in the eluted F(ab′)₂ with tris(2-carboxyethyl) phosphine (TCEP) releases Fd′ fragments having the second partner molecule attached. This Fd′ fragment can then be analyzed by mass spectrometry (STEP 8). Alternatively to TCEP, another reducing agent such as cysteamine hydrochloride can be used. Typical reducing conditions are 37° C. for 60 min or 60° C. for 30 min.

FIG. 2B shows dual capture of this disclosure applied to an ADC wherein a glycosylated (★) antibody has a first partner molecule (○) attached to its Fc fragment, a second partner molecule (●) attached to its Fd′ fragment, and a third partner molecule (□) attached to its light chain (LC). Analysis for transformation in the first and second partner molecules can be performed following STEPS 1-8 in the same manner as for FIG. 2A. Analysis for transformation in the third party molecule can be performed STEP 8, since the reduction of interchain disulfide bonds in F(ab′)₂ results in the formation of both LC and Fd′ fragments, each having attached its respective partner molecule and which can be analyzed by LC/HRMS in a single injection. Alternatively, the light chain can be analyzed per Kaur et al. 2013 or Darwish et al. 2017.

While FIGS. 2A and 2B symbolically show the first, second, and third (if present) partner molecules as different, they can be either the same or different. Even where the partner molecules are all the same, it may be desirable to analyze for transformation in the light chain, Fd′ fragment, and Fc fragment, to determine if the location of attachment has an effect on the occurrence or not of a transformation.

An ADC wherein the antibody has partner molecules attached to its light chain, Fd′ fragment, and Fc fragment can be made by a random conjugation method. A commonly used random conjugation technique entails modification of antibody lysine side chains with 2-iminothiolane to introduce a thiol group that can react with a maleimide terminated drug linker moiety, as shown below. Since lysines are found throughout an antibody, such random conjugation can introduce partner molecules into the light chain, the Fd′ fragment, and the Fc fragment.

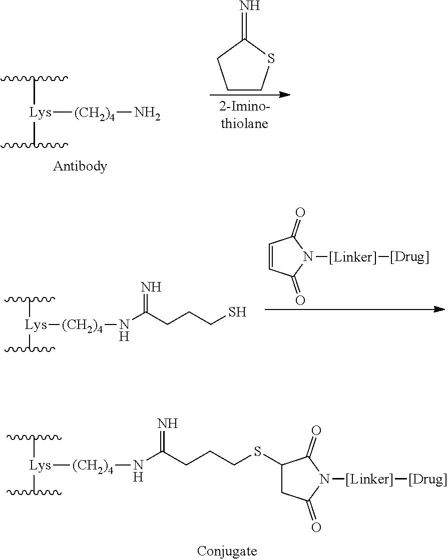

Conjugation of using the above lysine modification method will result in a random distribution of the same partner molecule/drug on the antibody, which can be symbolically represented as follows:

Those skilled in the art will appreciate that the depictions in FIGS. 1 and 2A-2B are illustrative, and not limitative. The ADC antibody is shown as a human antibody, but need not be so. It can be of other mammalian origin, such as from mouse, hamster, rabbit, or non-human primate. If non-human, the antibody can be chimeric or humanized. If the ADC antibody is non-human, then the specificities of the capture beads in STEPS 1 and 3 are altered accordingly, as are the digesting enzymes, mutatis mutandis. Also, actual DAR's may differ from those symbolically illustrated in the figures.

The DARs of the ADCs in FIGS. 1 and 2A-2B are shown as two, four, and six, respectively, but can be a higher or lower number. DAR will vary depending on the method of conjugation (e.g., random or site-specific) and in practice the experimentally measured DAR will usually be a non-integer number: a statistical average due to less than 100% efficiency in the conjugation process. Also, the depiction of the position of attachment of the partner molecules in FIGS. 1 and 2A-2B is figurative. Methods are known for attaching the partner moieties to various regions of an antibody, including the C-terminus, the N-terminus, the heavy or light chain variable regions, and the Fc fragment, and with different DAR values and site specificities.

Also, FIGS. 1 and 2A-2B show the ADC antibody as bearing glycan groups. Most native antibodies are glycosylated, with the glycan attached to asparagine 297 (N297 in Kabat numbering). However, antibodies used in ADCs do not need to be glycosylated. An ADC with glycan-free antibody can be obtained by treating the antibody with PNGase F prior to conjugation or by bioengineering the antibody with a site-specific mutation such as N297A, thus eliminating the N297 attachment site.

Antibodies that can be used in the ADC include those recognizing the following antigens: HER2, mesothelin, prostate specific membrane antigen (PSMA), CD19, CD22, CD30, CD70, B7H3, B7H4 (also known as O8E), protein tyrosine kinase 7 (PTK7), glypican-3, RG1, fucosyl-GM1, CTLA-4, and CD44. The antibody can be animal (e.g., murine), chimeric, humanized, or, preferably, human. The antibody preferably is monoclonal, especially a monoclonal human antibody. The preparation of human monoclonal antibodies against some of the aforementioned antigens is disclosed in Korman et al., U.S. Pat. No. 8,609,816 B2 (2013; B7H4); Rao-Naik et al., 8,097,703 B2 (2012; CD19); King et al., U.S. Pat. No. 8,481,683 B2 (2013; CD22); Keler et al., U.S. Pat. No. 7,387,776 B2 (2008; CD30); Terrett et al., U.S. Pat. No. 8,124,738 B2 (2012; CD70); Korman et al., U.S. Pat. No. 6,984,720 B1 (2006; CTLA-4); Korman et al., U.S. Pat. No. 8,008,449 B2 (2011; PD-1); Huang et al., US 2009/0297438 A1 (2009; PSMA); Cardarelli et al., U.S. Pat. No. 7,875,278 B2 (2011; PSMA); Terrett et al., U.S. Pat. No. 8,222,375 B2 (2012; PTK7); Harkins et al., U.S. Pat. No. 7,335,748 B2 (2008; RG1); Terrett et al., U.S. Pat. No. 8,268,970 B2 (2012; mesothelin); Xu et al., US 2010/0092484 A1 (2010; CD44); Deshpande et al., U.S. Pat. No. 8,258,266 B2 (2012; IP10); Kuhne et al., U.S. Pat. No. 8,450,464 B2 (2013; CXCR4); and Korman et al., U.S. Pat. No. 7,943,743 B2 (2011; PD-L1); the disclosures of which are incorporated herein by reference. The antibody can be trastuzumab, an anti-HER2 antibody.

Preferably the partner molecule a cytotoxic drug that can be used in cancer treatment by causing the death of cancer cells. Cytotoxic drugs that can be used in conjugates include the following types of compounds and their analogs and derivatives:
(a) enediynes such as calicheamicin (see, e.g., Lee et al., J. Am. Chem. Soc. 1987, 109, 3464 and 3466) and uncialamycin (see, e.g., Davies et al., WO 2007/038868 A2 (2007); Chowdari et al., U.S. Pat. No. 8,709,431 B2 (2012); and Nicolaou et al., WO 2015/023879 A1 (2015));
(b) tubulysins (see, e.g., Domling et al., U.S. Pat. No. 7,778,814 B2 (2010); Cheng et al., U.S. Pat. No. 8,394,922 B2 (2013); and Cong et al., U.S. Pat. No. 8,980,824 B2 (2015));
(c) DNA alkylators such as analogs of CC-1065 and duocarmycin (see, e.g., Yang et al., US 2018/0051031 A1 (2018); Boger, US 6,5458,530 B1 (2003); Sufi et al., U.S. Pat. No. 8,461,117 B2 (2013); and Zhang et al., U.S. Pat. No. 8,852,599 B2 (2014));
(d) epothilones (see, e.g., Vite et al., US 2007/0275904 A1 (2007) and US RE42930 E (2011));
(e) auristatins (see, e.g., Senter et al., U.S. Pat. No. 6,844,869 B2 (2005) and Doronina et al., U.S. Pat. No. 7,498,298 B2 (2009));
(f) benzodiazepine dimers (see, e.g., Zhang et al., U.S. Pat. No. 9,527,871 B2 (2016); Zhang et al., U.S. Pat. No. 9,688,694 B2 (2017); McDonald et al., U.S. Pat. No. 9,526,801 B2 (2016); Howard et al., US 2013/0059800 A1 (2013); US 2013/0028919 A1 (2013); and WO 2013/041606 A1 (2013)); and
(g) maytansinoids such as DM1 and DM4 (see, e.g., Chari et al., U.S. Pat. No. 5,208,020 (1993) and Amphlett et al., U.S. Pat. No. 7,374,762 B2 (2008)).

In addition to anti-cancer drugs, other partner molecules, such as adjuvants, radioisotopes, reporter groups, fluorescent labels, peptides, and the like can be used, where they could undergo a transformation while still attached to the ADC, which might bear monitoring.

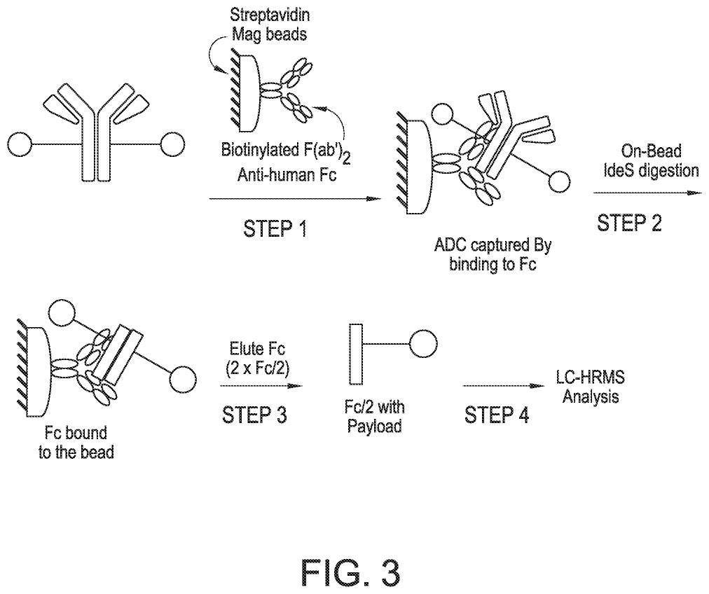

FIG. 3 is a comparative figure showing a single capture method for analyzing an ADC having a partner molecule attached to the Fc fragment. STEP 1 differs from those of FIGS. 1 and 2A-2B in that the specificity of the capture beads is for the Fc fragment of the ADC antibody, and not its F(ab′)₂ region. In theory, IdeS digestion followed by elution could afford Fc fragments with an attached partner molecule, which can then be analyzed by mass spectrometry. However, as will be shown below, an Fc-capture seems to create steric hindrance blocking a complete digestion by the protease, and therefore the scheme of this figure is less desirable than those of FIGS. 1 and 2.

### General Procedures

The following general procedures were employed.

### Chemicals and Reagents

Streptavidin Mag Sepharose Beads and HBS-EP buffer (0.01 M HEPES pH 7.4, 0.15 M NaCl, 3 mM EDTA, 0.005% v/v Surfactant P20) were obtained from GE Healthcare Life Sciences (PA, USA). Biotin-SP (long spacer) AffiniPure F(ab′)₂ Fragment Goat Anti-Human IgG Fcγ Fragment Specific, and F(ab′)₂ Fragment Specific capture reagents were purchased from Jackson ImmunoResearch (PA, USA). IdeS Protease (FabRICATOR™) and EndoS2 (GlycINATOR™) were purchased from Genovis (MA, USA), and Peptide N-glycosidase F (PNGase F) was purchased from Promega (Madison, WI). LC-MS grade water and acetonitrile were purchased from Burdick and Jackson (MI, USA). Tris(2-carboxyethyl) phosphine hydrochloride (TCEP-HCl) and LC-MS grade formic acid was obtained from Thermo Fisher Scientific (IL, USA).

### Preparation of ADCs

Transglutaminase-mediated conjugation was conducted as follows. The purified antibodies (˜2-5 mg/ml in 20 mM Tris pH8 buffer) were reacted with 10-fold molar (per site) excess of the amine donor in the presence of 0.2 molar recombinant bacterial transglutaminase per antibody. The reaction was allowed to proceed overnight at 37° C. with continuous gentle mixing. The conjugated product was purified by protein A HP SpinTrap or HiTrap S column.

Dual drug conjugation was prepared as follows. Unpaired cysteine introduced antibody was subjected to mild reduction in PBS pH7.4/2 mM EDTA at 37° C. by the addition of 30-fold molar (per antibody) excess TCEP for 2 hrs until separation of the light chains and heavy chains is observed on RP-UPLC (Agilent AdvancedBio Diphenyl 2.1×150 mm). The mobile phase consisted of 0.1% TFA in water (A) and 0.1% TFA in acetonitrile (B). The system was operated at a flow rate of 1 ml/min. The gradient condition was as follows: 0-1 min., 20-30% B; 1-6 min, 30-42%, 6.05-6.5 min, 42-95%. Wavelength 280 nM was used to monitor the reduction. TCEP was removed by Zeba column. Buffer was exchanged to PBS pH6.8/2 mM EDTA. Re-oxidation of mAb was conducted with dhAA. The reoxidation was monitored by RP-UPLC by following the disappearance of light and heavy chain peaks and reappearance of the intact Ab peak. The re-formation of the interchain disulfide was conducted with incubation with dhAA at 50-fold excess per antibody. The mixture was incubated at 25° C. for 3 hrs. The maleimide-linked reagent was incubated with the activated antibody for 1 hr at 25° C. The antibody conjugate was purified on HiTrap S column to remove excess reagents. The purified antibody was dialyzed into 20 mM tris pH8 buffer, followed by standard bTGase conjugation protocol as described above.

Formation of drug-conjugates were confirmed by LC-MS analysis. The samples were diluted to 1 mg/ml in 100 mM Tris pH 7.5. 20 μl of sample was reduced by adding 2 μl of 0.5M DTT or TCEP. The samples were analyzed by LC-MS using Agilent 1290 Infinity UPLC coupled to a 6530 Accurate-Mass Q-TOF. The analytical column used was waters BEH C4 column, 1.7 μm, 2.1 mm×50 mm held at 60° C. The mobile phase consisted of 0.1% formic acid in water (A) and 0.1% formic acid in acetonitrile (B). The system was operated at a flow rate of 200 uμ/min. The gradient condition was as follows: 0-2 min., held at 27% B; 2-9 min., slow ramp from 27-37% B; 9-9.5 min., linear ramp from 37-90% B; 9.5-12.3 min., held at 90% B. The MS settings were as follows: Polarity=Positive, Capillary Voltage=4.2 kV, Sample Cone=40 V, Source Offset=15 V, Source Temperature=140° C., Desolvation Temperature=325° C. The data acquisition range was 900-3200 m/z. Deconvolution was done using Agilent MassHunter Walkup.

### In Vitro and In Vivo Samples

For in vitro serum stability assessment, ADCs were added to 500 μl serum from SCID mice (BioIVT, Westbury, NY) at a final concentration of 50 μg/mL and incubated at 37° C. 100 μL serum aliquots were taken at selected intervals post incubation and stored at −80° C. until analysis. For in vivo stability/biotransformation assessment, SCID mice were dosed with a single intraperitoneal or intravenous injection of ADC. Blood samples were collected from three mice at different time points post dosing and further centrifuged to obtain serum. The serum samples were stored at −80° C. until the final sample preparation.

### Preparation of Magnetic Capture Beads

Streptavidin Mag Sepharose Beads (550 μL) were transferred into a 2-mL Eppendorf tube. The beads were washed thrice with 1000 μL of HBS-EP buffer and resuspended in 1.6 mL of HBS-EP. 120 μg of capture reagent: Anti-Human IgG F(ab′)₂ Fragment Specific (Capture Reagent-1) or Anti-Human IgG Fcγ Fragment Specific (Capture Reagent-2) was then added to the beads and incubated for 2 h on a Hula mixer (Thermo Fisher Scientific, IL, USA) to prepare anti-human F(ab′)₂ and anti-human Fc coated beads, respectively. After incubation, the beads were washed thrice with 1000 μl of HBS-EP buffer and resuspended in 1 ml of HBS-EP.

### Sample Preparation

Serum samples containing these ADCs were incubated with anti-human F(ab′)₂ coated beads (Capture-1) and HBS-EP buffer as already described above. After the ADC was immobilized on the beads, the beads were then washed 3× with 500 μL of HBS-EP buffer and transferred to a deep-well plate containing 300 μL of HBS-EP and 100 units each of IdeS and PNGase F (latter optional depending on whether the ADC is glycosylated). The deep-well plate was incubated at 37° C. for 1.5 h on a Thermomixer C at 1000 rpm to release the deglycosylated Fc fragment. At the end of this enzymatic incubation, the anti-human F(ab′)₂ coated beads were removed from the incubation/digest plate. The deglycosylated Fc fragment present in the incubation/digest plate was captured by addition of 50 μl of anti-human Fc coated beads (Capture-2) and further incubation at room temperature for 1 hour at 1200 rpm. The Fc fragment bound to the beads was eluted with 75 μl of elution solvent (1% formic acid in 25% Acetonitrile), after the beads were washed with HBS-EP and water (two times each), and subsequently analyzed by LC-MS. For Fc conjugated ADCs, the anti-human F(ab′)₂ coated beads (Capture-1) were discarded.

### Mass Spectrometry

Samples were analyzed by middle-up LC-MS using an Acquity I-Class UPLC coupled to a Xevo G2-XS TOF (Waters Corp, MA, USA). The analytical column used was a Waters BEH C4, 1.8 μm, 2.1 mm×50 mm held at 80° C. The mobile phase consisted of 0.1% formic acid in water (A) and 0.1% formic acid in acetonitrile (B). The system was operated at a flow rate of 400 μL/min.

The gradient condition was as follows: 0-2 min, held at 10% B; 2-4 min, linear ramp from 10-25% B; 4-10 min, slow ramp from 25-37% B; 10-15 min, a zigzag gradient of 90-10-90-10-90-10, followed by 15-17 min, equilibration at 10% B

The MS settings were as follows: Polarity=Positive, Capillary Voltage=3 kV, Sample Cone=40 V, Source Offset=30 V, Source Temperature=140° C., Desolvation Temperature=350° C., Cone Gas flow=0 L/h, Desolvation Gas Flow=1000 L/h. The data acquisition range was 500-3000 m/z in Masslynx (Version 4.1).

Deconvolution was done using MaxENT 1 algorithm of Masslynx software (Version 4.1).

### EXAMPLES

The methods of this disclosure can be further understood by reference to the following examples, which are provided by way of illustration and not of limitation.

### Example 1

Transglutaminase can form an amide bond between the carboxamide side chain of a glutamine (the amine acceptor) in a first protein and the E-amino group of a lysine (the amine donor) in a second protein, in a transamidation reaction. Specificity-wise, it is selective regarding the glutamine residue, requiring that it be located in a flexible part of a protein loop and flanked by particular amino acids. Conversely, transglutaminase is permissive regarding the lysine residue: it accepts an amino group from a non-protein source, such an alkyleneamino compound, as a lysine E-amino surrogate.

Antibodies of the IgG isotype have many glutamines-nine or more in the heavy chain constant region alone, the exact number depending on isotype. However, none of them are transglutaminase-reactive in a native antibody-that is, they are not transamidated by transglutaminase. Normally, an antibody is glycosylated at asparagine 297 (N297) of the heavy chain (N-linked glycosylation). Jeger et al., Angew. Chem. Int. Ed. 2010, 49, 9995, discovered that deglycosylation of the antibody, either by eliminating the glycosylation site through an N297A site-specific substitution or post-translation enzymatic deglycosylation with an enzyme such as PNGase F unblocks nearby glutamine 295 (Q295) and renders it transglutaminase-reactive. (References to amino acid positions in an antibody constant region employ numbering per the EU index as set forth in Kabat et al., “Sequences of proteins of immunological interest,” 5th ed., Pub. No. 91-3242, U.S. Dept. Health & Human Services, NIH, Bethesda, Md., 1991; hereinafter “Kabat.”) They further showed that an N297Q substitution not only eliminates glycosylation, but also introduces a second glutamine residue, at position 297, that is an amine acceptor. Thus, simple deglycosylation generates two transglutaminase-reactive glutamine residues per antibody (one per heavy chain, at Q295), while an N297Q substitution generates four transglutaminase-reactive glutamine residues (two per heavy chain, at Q295 and Q297). Thus, antibodies with N297A or N297Q site specific substitutions are frequently used for the preparation of ADCs via transglutaminase-mediated transamidation.

In this example, an antibody-drug conjugate, designated ADC-5, was prepared from an anti-mesothelin antibody (see, e.g., Terrett et al., U.S. Pat. No. 8,268,970 B2 (2012)) having a glutamine-containing peptide extension at the C-terminus of each heavy chain. This glutamine, being exposed, was transglutaminase-reactive. Since the antibody retained the native N297, it was glycosylated and its other glutamines were not transglutaminase-reactive. This antibody was conjugated to a tubulysin analog via a linker having an extended alkyl amino group to serve as a lysine surrogate amine donor (Young et al., U.S. application Ser. No. 16/437,047, filed Jun. 11, 2019). With each extension providing one reactive glutamine, the theoretical DAR is two. The structure of the resulting ADC-5 can be represented by formula (A) while the detailed structure of the tubulysin analog partner molecule and linker are shown by formula (B), where Ab denotes the antibody.

Tubulysin is an anti-cancer natural product whose mechanism of action is tubulin disruption, thus preventing mitosis. Both tubulysin and the analog have an acetate group (arrow in formula (B)), the hydrolysis of which leads to loss of activity.

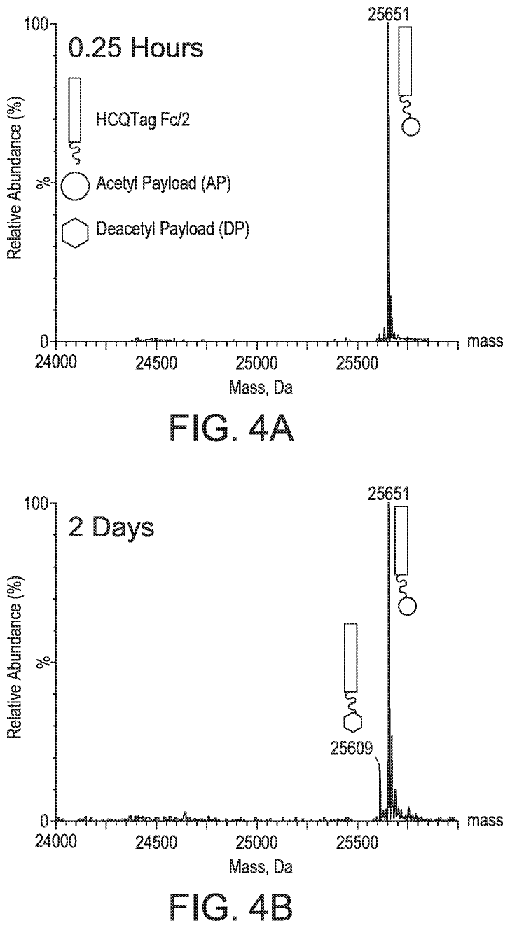

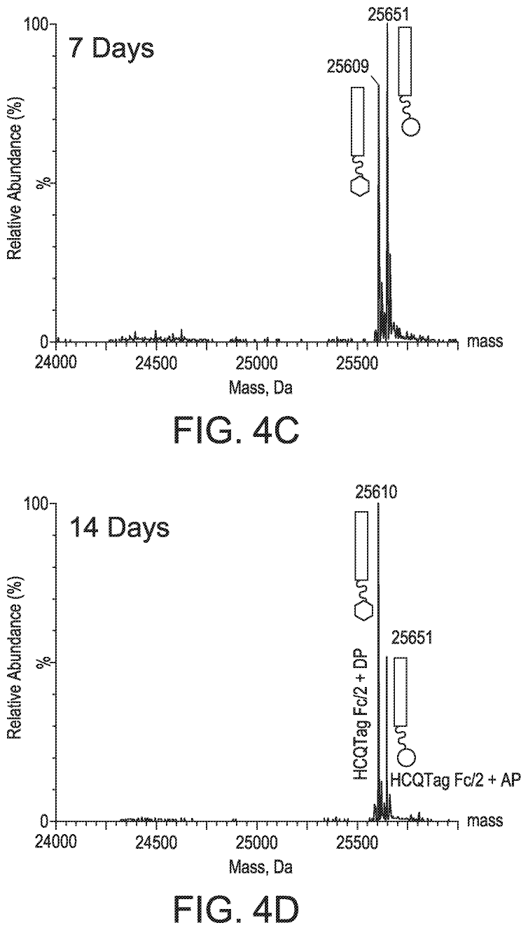

Employing the dual capture method disclosed shown in FIG. 1, the hydrolysis of the acetate group was monitored over time. The results are shown in FIGS. 4A (0.25 hours), 4B (2 days), 4C (7 days), and 4D (14 days).

### Example 2

This example relates to a double antibody-drug conjugate, designated ADC-6,wherein the antibody has a first partner molecule attached to its Fc fragment and a second partner molecule attached to its Fab region. The antibody is an anti-mesothelin antibody. The antibody has a glutamine-containing peptide “tag” in its Fab region (one per heavy chain), thus providing a total of two reactive glutamines. Consequently, ADC-6 has a theoretical DAR of 4 and its structure can be schematically represented by the formula below.

The first partner molecule is the same tubulysin analog of the previous example, but having a different linker, one adapted for conjugation by maleimide addition chemistry, with the cysteine used for the addition chemistry introduced via site-specific substitution. See, e.g., Cheng et al., U.S. Pat. No. 8,394,922 B2 (2013). The detailed first partner molecule structure and its linker attachment to the antibody can be represented by formula (D) below. There are two sites susceptible to transformation: hydrolysis of the succinimide group (arrow 1) and deacetylation of the acetate group (arrow 2).

The second partner molecule is a DNA minor groove binder of the type described in McDonald et al., US 2018/0110873 A1 (2018). It is attached Fab region by transglutaminase-mediated conjugation, as described above. Its structure is represented by formula (C) below.

One transformation that can be monitored is peptide cleavage at the position indicated by the arrow.

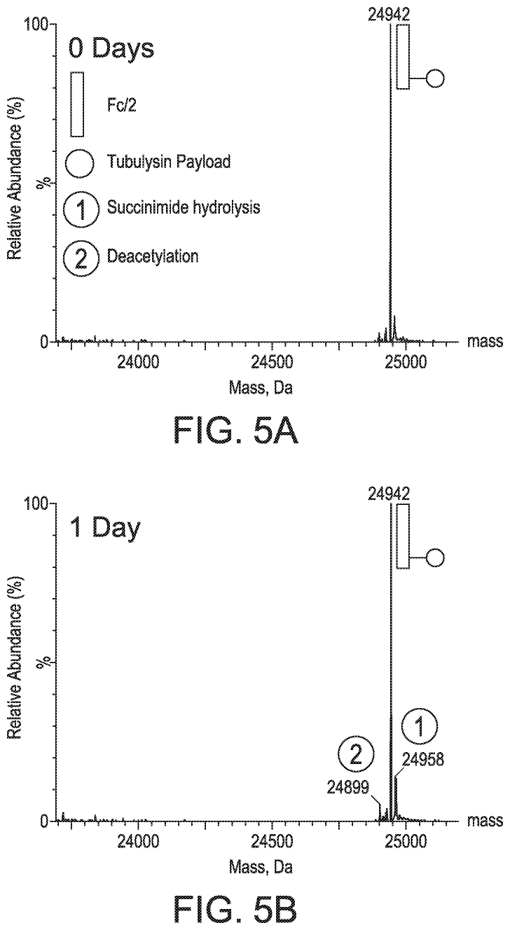

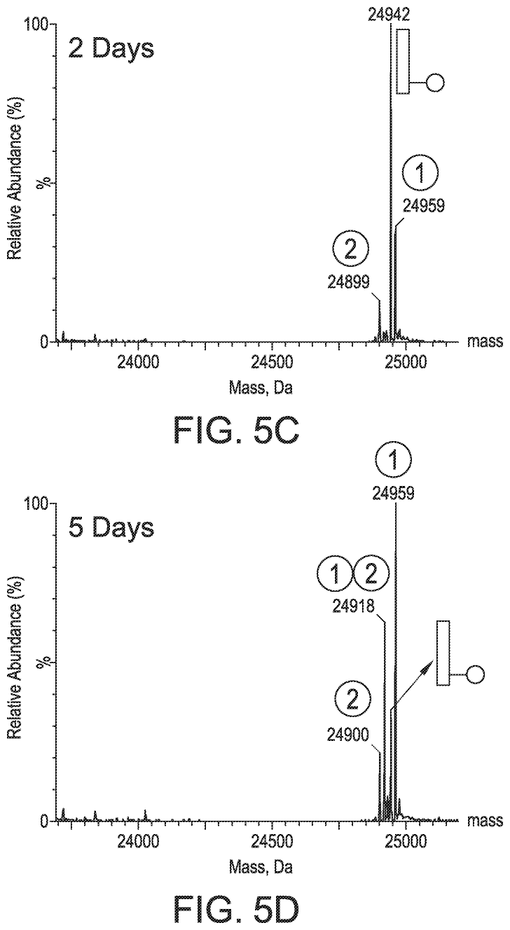

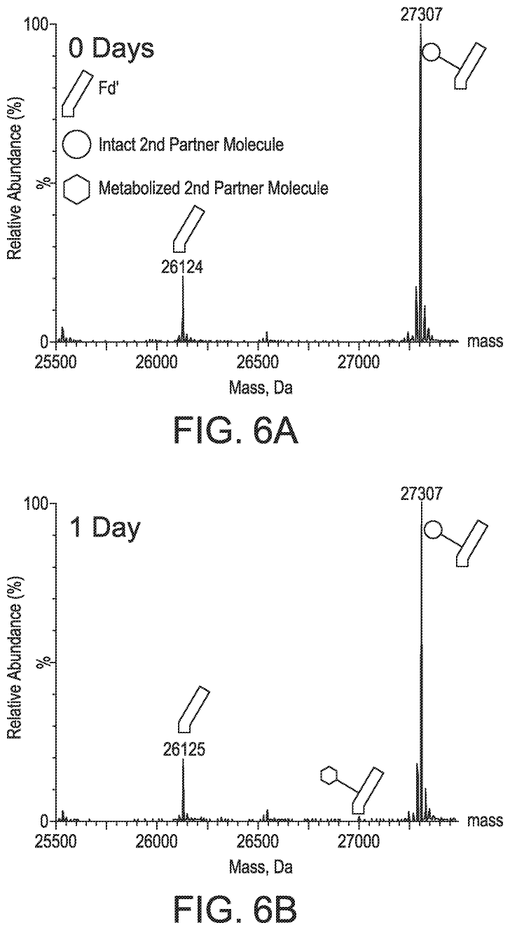

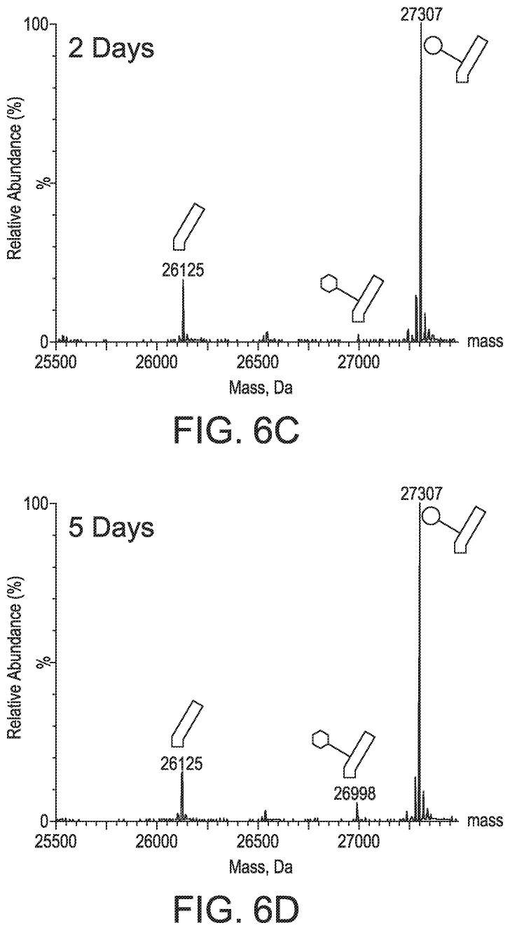

ADC-6 was processed by the scheme of FIG. 2A to afford an Fc fragment having the first partner molecule attached and an Fd′ fragment having the second partner molecule attached. Each of the fragments was then analyzed by LC-HRMS. FIGS. 5A, 5B, SC, and 5D show how both the succinimide hydrolysis and deacetylation can be monitored over five days in the Fc fragment. FIGS. 6A, 6B, 6C, and 6D show how the peptide cleavage in the second partner molecule can be followed over the course of the same five days.

### Example 3

In this Example, the antibody used was an anti-fucosyl GM1 one and had an N297A site specific substitution, thus abrogating glycosylation. Transglutaminase mediated conjugation with the same partner molecule/linker as in Example 1 afforded an ADC—designated ADC-3—with a theoretical DAR of two, with the drug linked to Q295 as discussed above. Its structure can be represented schematically by formula (E). The detailed structure of its partner molecule is the same as in formula (B), including the deacetylation site.

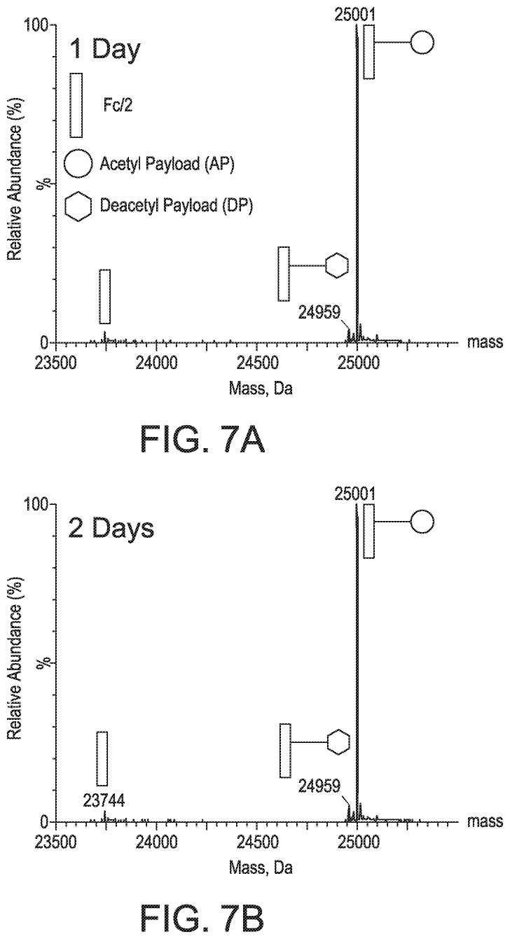

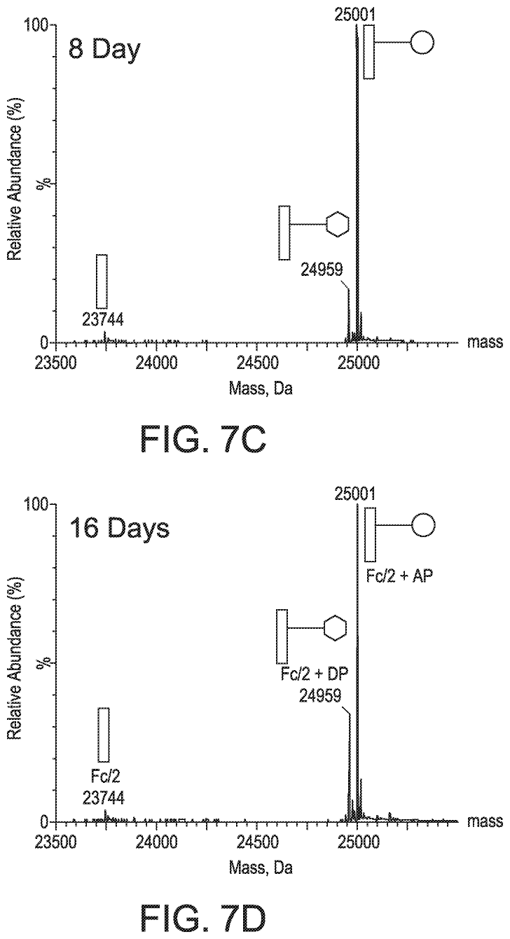

ADC-3 was processed using the single capture scheme of FIG. 3. The mass spec analysis results are shown in FIGS. 7A-7D.

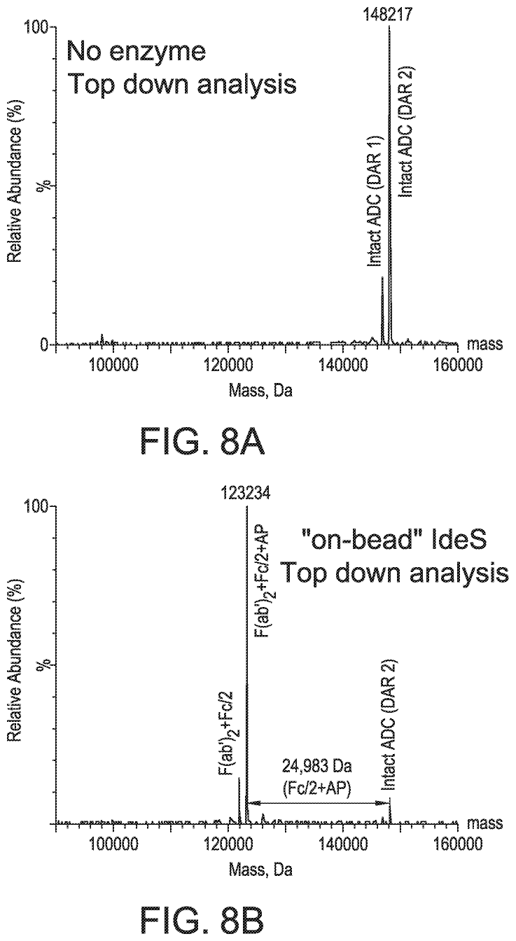

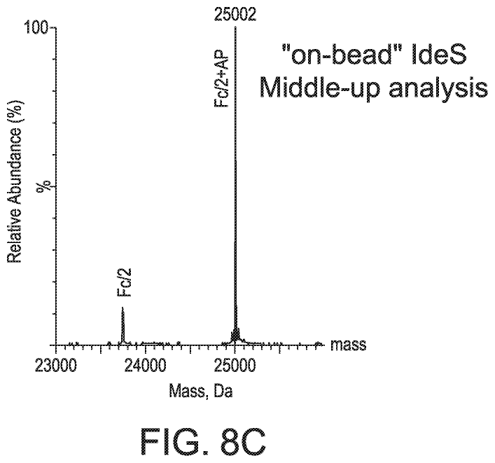

However, upon closer inspection, it was determined that there was incomplete digestion by IdeS, as shown in FIGS. 8A-8C. (In these figures “top down analysis” means that analysis was performed on the intact ADC, MW ˜150,000 Da, while “middle-up analysis” means the ADC was digested with IdeS to generate fragments of ˜25,000 Da.) We estimate that the efficiency of digestion by IdeS was only about 50%. We hypothesize that the inefficiency is due to steric hindrance from the interaction of the ADC-3 Fc fragment with the capture reagent, preventing IdeS access to the cleavage site below the hinge.

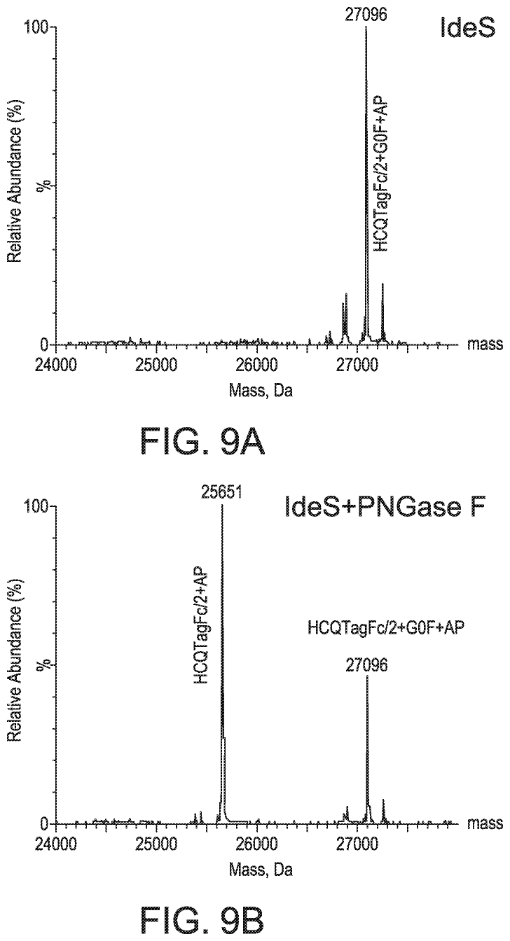

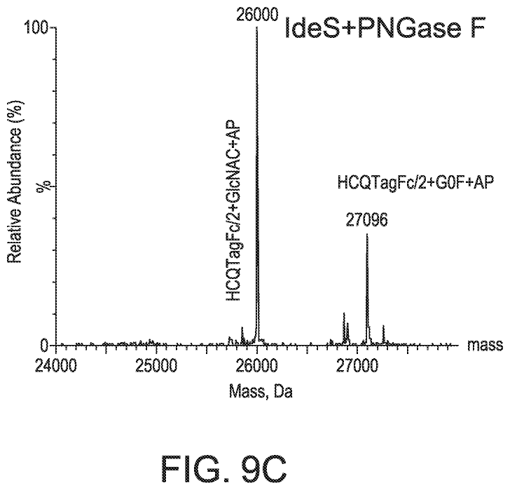

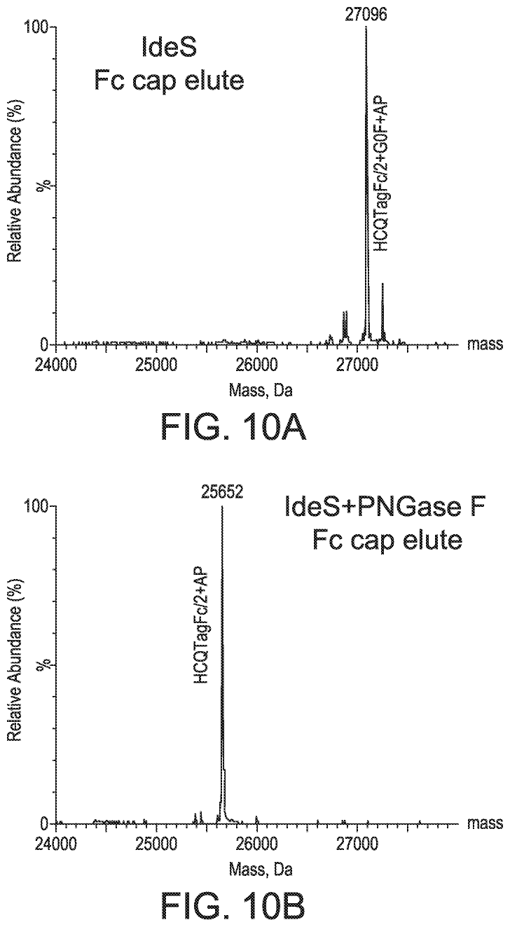

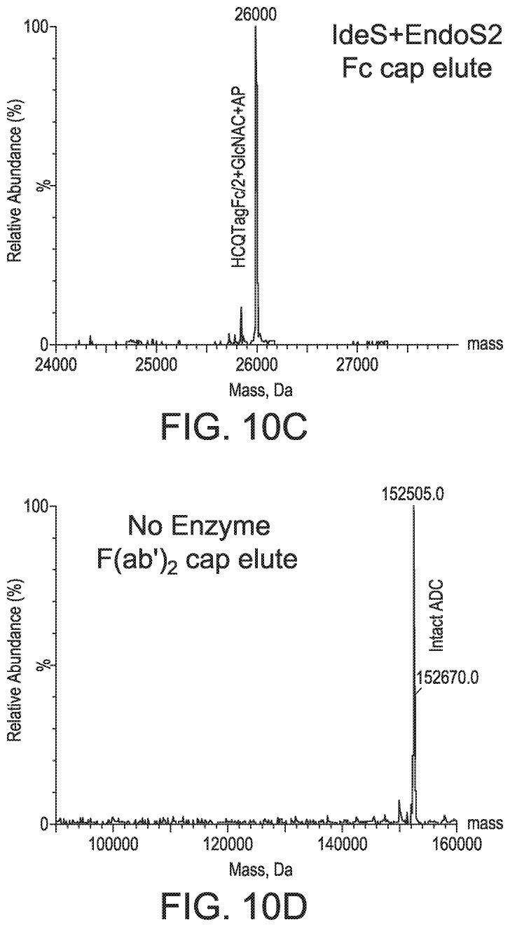

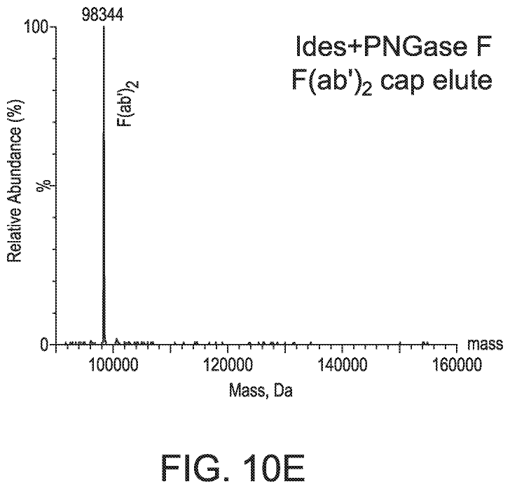

Delving further into this hypothesis, we subjected ADC-5 to the same single capture method of FIG. 3. Mono capture with Fc followed by “on-bead” digestion with IdeS+PNGase F or IdeS+Endo S2 showed incomplete removal of N-glycan due to steric hindrance (FIGS. 9A-9C). When ADC-5 subjected to dual capture, i.e., first captured by anti-human F(ab′)₂ and “on-bead” digestion with IdeS+PNGase For IdeS+Endo S2, then followed by dual capture of the released Fc, N-glycans were effectively removed (FIGS. 10A-10E)

These results show that, even though the single capture method is able to provide information about transformation on the partner molecule, it is less efficient than the dual capture method of either FIG. 1 or FIGS. 2A-2B.

### Example 4

The lysine/2-iminothiolane modification plus maleimide addition random conjugation procedure described above was used to make an ADC in which the partner molecule was a tubulysin analog and the antibody was an anti-mesothelin antibody (Cheng et al., U.S. Pat. No. 8,394,922 B2 (2013)). The ADC structure can be represented by the formula below, where Ab denotes the antibody and n denotes the average DAR, which was 3. This ADC will be referred to as ADC-7.

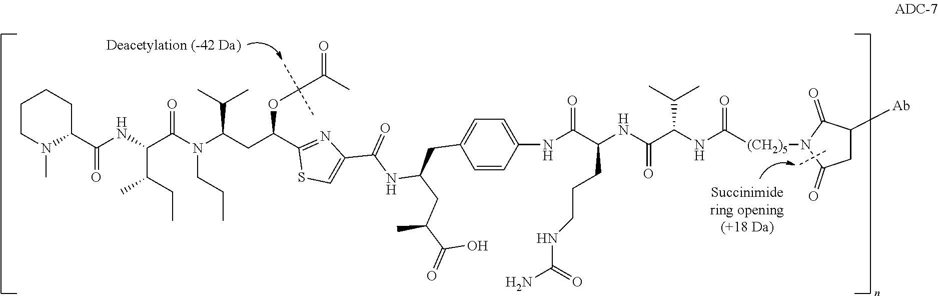

Because of the nature of the conjugation process, the partner molecules are distributed randomly throughout the antibody wherever lysines have been modified by the 2-iminothiolane, including at the light chain (LC), the Fd′ fragment, and the Fc fragment. Symbolically, an ADC wherein the LC, Fd′ fragment, and the Fc fragment each bear a partner molecule (○) can be represented as follows:

The transformations of the partner molecule and the linker that were monitored are acetate group hydrolysis (deacetylation; −42 Da change in mass) and of the succinimide hydrolysis (ring opening; +18 Da change in mass), as indicated in the formula above. Also monitored was the situation in which both deacetylation and succinimide hydrolysis occurred (net −24 Da change in mass).

ADC-7 was incubated in SCID mouse serum for 96 hr. Aliquots were taken at start (0 Days), 2 days, and 4 days incubation time.

Analysis for transformation was performed per the scheme of FIG. 2A, understanding that the three symbols ●, ○, and □ represent in this instance identical partner molecules. ADC-7 was first captured from serum using anti-human (F(ab′)₂ coated magnetic beads (STEP 1). The captured ADC-7 was digested with IdeS and PNGase F to release the deglycosylated, partner molecule-containing, Fc fragment (STEP 2). This Fc fragment was then captured on anti-human Fc coated beads (STEP 3). The F(ab′)₂ fragment still bound to the beads after STEP 2 was eluted (STEP 6) and then reduced with TCEP (STEP 7) to release Fd′ and Fc fragments, each containing bound partner molecule. The bead-bound Fc fragment from STEP 3 was eluted (STEP 4). The Fc fragment, the Fd′ fragment, and the light chain were each analyzed by liquid chromatography/high resolution mass spectrometry (LC-HRMS) in STEPS 5, and 8, respectively, for deacetylation and/or succinimide hydrolysis.

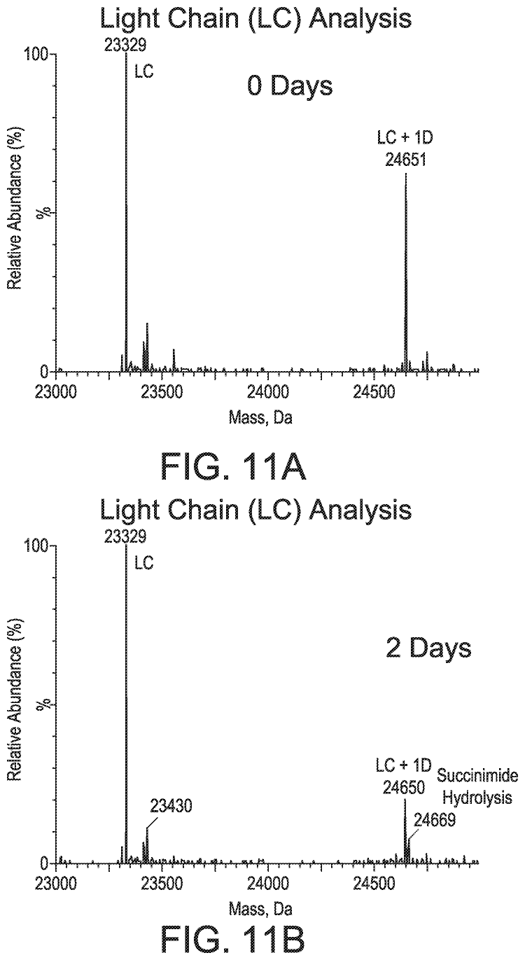

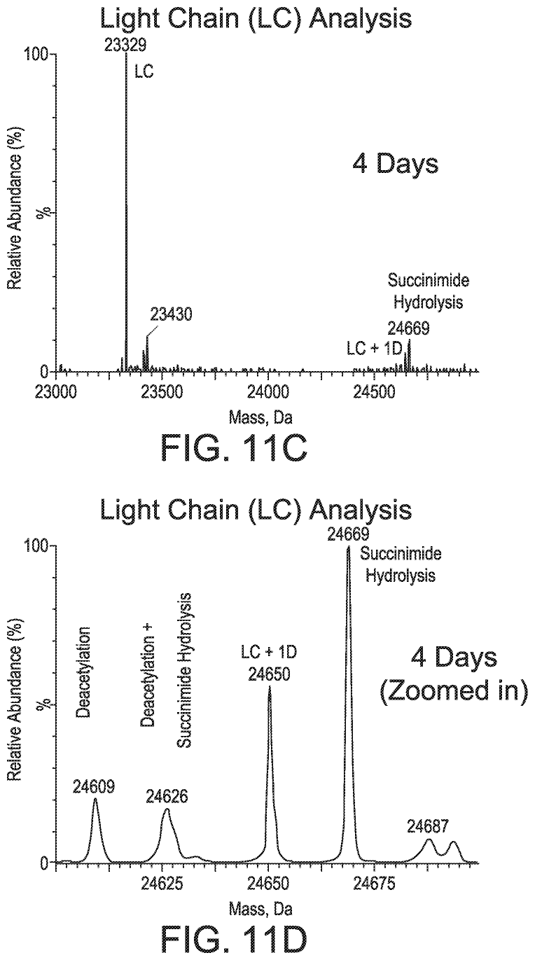

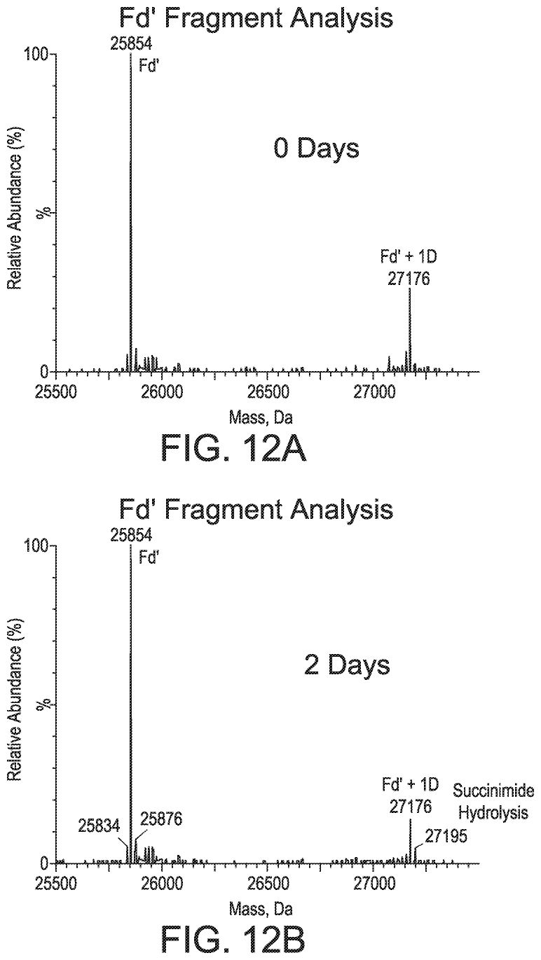

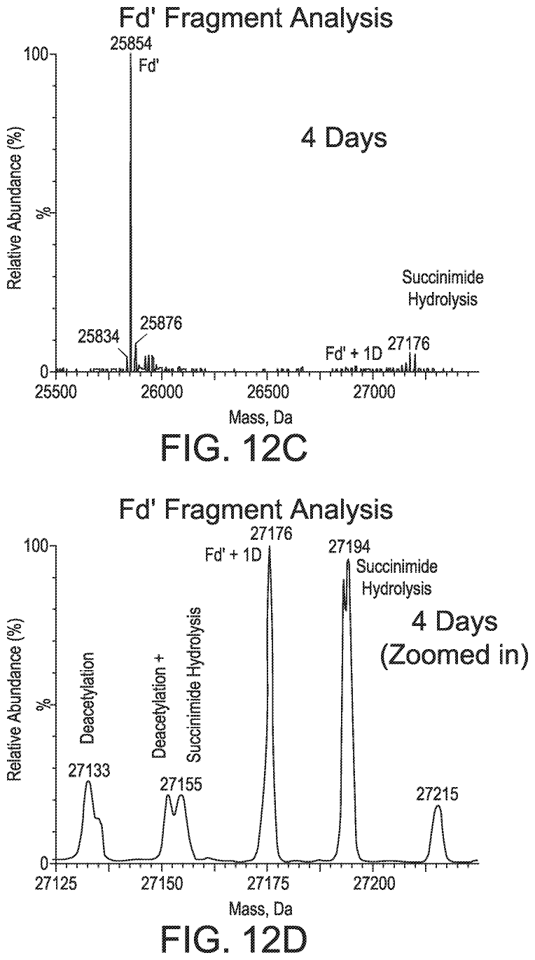

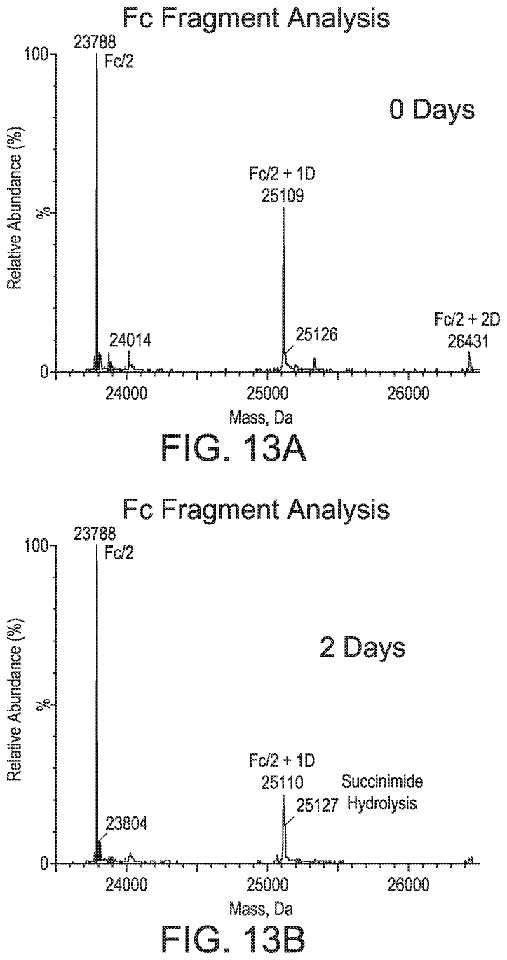

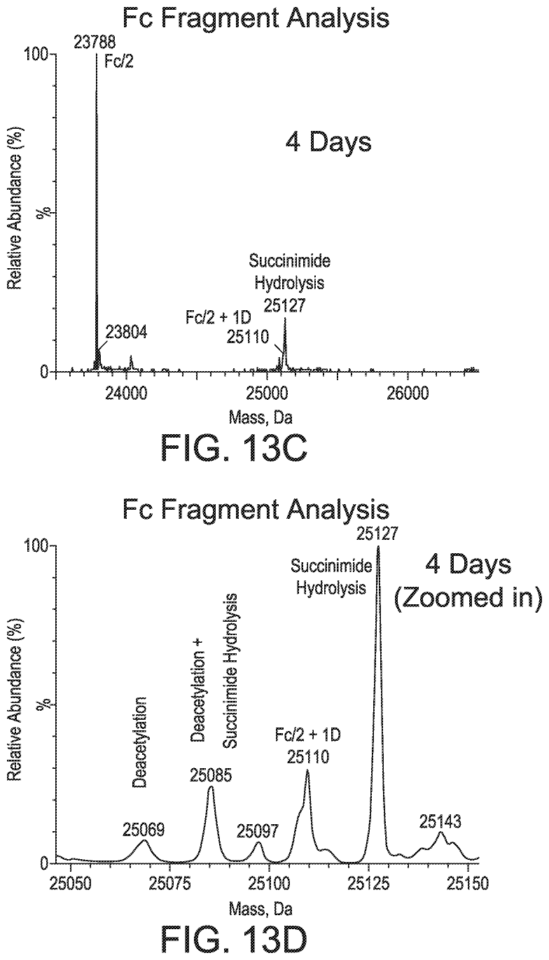

FIGS. 11A, 11B, and 11C show the analytical results at 0, 2, and 4 Days, respectively, for the light chain. FIG. 11D is a zoomed in view of the 4 Days data clustered around the 24,650 Da peak of the light chain (LC) with one partner molecule/drug attached (“LC+1D”). Peaks corresponding to succinimide hydrolysis/ring opening (+18 Da), deacetylation (−42 Da), and succinimide hydrolysis plus deacetylation (−24 Da) are visible. There also is a decrease in the relative intensity of the LC+1D peak, indicating deconjugation.

FIGS. 12A, 12B, and 12C show the analytical results at 0, 2, and 4 Days, respectively, for the Fd′ fragment. FIG. 12D is a zoomed in view of the 4 Days data around the 27,176 Da peak for the Fd′ fragment with one partner molecule/drug attached (“Fd′+1D”). Again, +18 Da, −42 Da, and −24 Da peaks are visible, along with a decrease in the relative intensity of the Fd′+1D peak indicating deconjugation.

FIGS. 13A, 13B, and 13C show the analytical results at 0, 2, and 4 Days, respectively, for the Fc fragment. FIG. 13D is a zoomed in view of the 4 Days data around the 25,110 Da peak for an Fc fragment with one partner molecule/drug attached (“Fc/2+1D”). Again, +18 Da, −42 Da, and −24 Da peaks are visible, along with a decrease in the relative intensity of the Fc/2 +1D peak indicating deconjugation.

### DEFINITIONS

“Antibody” means whole antibodies and any antigen binding fragment (i.e., “antigen-binding portion”) or single chain variants thereof. A whole, or full length, antibody is a protein comprising at least two heavy (H) chains and two light (L) chains inter-connected by disulfide bonds. Each heavy chain comprises a heavy chain variable region (V_{H}) and a heavy chain constant region comprising three domains, C_{H1}, C_{H2} and CH₃. Each light chain comprises a light chain variable region (V_{L} or Vₖ) and a light chain constant region comprising one single domain, C_{L}. The V_{H} and V_{L} regions can be further subdivided into regions of hypervariability, termed complementarity determining regions (CDRs), interspersed with more conserved framework regions (FRs). Each V_{H} and V_{L} comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (Clq) of the classical complement system. An antibody is said to “specifically bind” to an antigen X if the antibody binds to antigen X with a K_{D} of 5×33 10⁻⁸ M or less, more preferably 1×10⁻⁸ M or less, more preferably 6×10⁻⁹ M or less, more preferably 3×10⁻⁹ M or less, even more preferably 2×10⁻⁹ M or less. The antibody can be chimeric, humanized, or, preferably, human. The heavy chain constant region can be engineered to affect glycosylation type or extent, to extend antibody half-life, to enhance or reduce interactions with effector cells or the complement system, or to modulate some other property. The engineering can be accomplished by replacement, addition, or deletion of one or more amino acids or by replacement of a domain with a domain from another immunoglobulin type, or a combination of the foregoing.

“Antigen binding fragment” and “antigen binding portion” of an antibody (or simply “antibody portion” or “antibody fragment”) mean one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fab' fragment, which is essentially an Fab with part of the hinge region (see, for example, Abbas et al., Cellular and Molecular Immunology, 6th Ed., Saunders Elsevier 2007); (iv) a Fd fragment (also called n Fd′ fragment) consisting of the V_{H} and C_{H1} domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Preferred antigen binding fragments are Fab, F(ab′)₂, Fab′, Fv, and Fd fragments. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” of an antibody.

Cleavage of an antibody above (i.e., on the amine terminal side) of the hinge disulfides by an enzyme such as papain releases two Fab fragments, each consisting of a light chain and the V_{H} and C_{H1} domains of a heavy chain, which are joined by disulfide bonds. Reduction of the disulfide bonds in an Fab fragment causes separation of the light chain and the V_{H}/C_{H1} chains, the latter being referred to as an Fd′ (or Fd) fragment. If, however, an antibody is cleaved below (i.e., on the carboxy terminus side) by an enzyme such as pepsin, the result is an F(ab′)₂ fragment in which two Fab fragments are joined by the hinge disulfide bonds. In turn, each half of the F(ab′)₂ fragment comprises the light chain and the Fd′ fragment of the heavy chain, joined by disulfide bonds.

Unless indicated otherwise-for example by reference to the linear numbering in a SEQ ID NO: listing—references to the numbering of amino acid positions in an antibody heavy or light chain variable region (V_{H} or V_{L}) are according to the Kabat system (Kabat et al., “Sequences of proteins of immunological interest, 5th ed., Pub. No. 91-3242, U.S. Dept. Health & Human Services, NIH, Bethesda, Md., 1991, hereinafter “Kabat”) and references to the numbering of amino acid positions in an antibody heavy or light chain constant region (C_{H1}, C_{H2}, C_{H3}, or C_{L}) are according to the EU index as set forth in Kabat. See Lazar et al., US 2008/0248028 A1, the disclosure of which is incorporated herein by reference, for examples of such usage. Further, the ImMunoGeneTics Information System (IMGT) provides at its website a table entitled “IMGT Scientific Chart: Correspondence between C Numberings” showing the correspondence between its numbering system, EU numbering, and Kabat numbering for the heavy chain constant region.

An “isolated antibody” means an antibody that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

“Monoclonal antibody” or “monoclonal antibody composition” means a preparation of antibody molecules of single molecular composition, which displays a single binding specificity and affinity for a particular epitope.

“Human antibody” means an antibody having variable regions in which both the framework and CDR regions (and the constant region, if present) are derived from human germline immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human antibody” does not include antibodies in which CDR sequences derived from the germline of another mammalian species, such as a mouse, have been grafted onto human framework sequences.

“Human monoclonal antibody” means an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

Unless specifically stated otherwise herein, references made in the singular may also include the plural. For example, “a” and “an” may refer to either one, or one or more.

### REFERENCES

Full citations for the following references cited in abbreviated fashion by first author (or inventor) and date earlier in this specification are provided below. Each of these references is incorporated herein by reference for all purposes.
Cheng et al., U.S. Pat. No. 8,394,922 B2 (2013).
Darwish et al., US 2017/0370906 A1 (2017).
Gerber et al., Nat. Prod. Rep. 2013, 30, 625, “The antibody-drug conjugate: an enabling modality for natural product-based cancer therapies.”
Jeger et al., Angew. Chem. Int. Ed. 2010, 49, 9995.
Kaur et al., U.S. Pat. No. 8,541,178 B2 (2013).
Young et al., U.S. application Ser. No. 16/437,047, filed Jun. 11, 2019.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

What is claimed is:

1. A method for analyzing whether, in an antibody-drug conjugate (ADC) having a partner molecule attached to the Fc fragment of the antibody, a transformation has occurred in the partner molecule, comprising the steps of:
a. capturing the ADC on first beads having an affinity for the F(ab′)₂ region of the antibody;
b. digesting the ADC captured on the first beads in a digestion medium comprising a protease that cleaves the antibody into an F(ab′)₂ fragment and an Fc fragment having the partner molecule attached thereto and optionally a deglycosylating enzyme, whereby the F(ab′)₂ fragment remains attached to the first beads and the Fc fragment is released;
c. capturing the released Fc fragment having the partner molecule attached thereto on second beads having an affinity for the Fc fragment;
d. eluting with an acidic eluent the Fc fragment having the partner molecule attached thereto from the second beads;
and
e. obtaining a mass spectrometric analysis of the eluted Fc fragment having the partner molecule attached thereto, to determine if a transformation occurred in the partner molecule.

2. A method according to claim 1, wherein the antibody in the ADC is a human antibody.

3. A method according to claim 2, wherein the protease is IdeS.

4. A method according to claim 2, wherein the antibody is glycosylated.

5. A method according to claim 4, wherein the digestion medium comprises a deglycosylating enzyme selected from the group consisting of PNGase and EndoS2.

6. A method according to claim 2, wherein the antibody lacks glycan groups.

7. A method according to claim 1, wherein the first beads comprise a streptavidin coating and a biotinylated F(ab′)₂ fragment having affinity for the F(ab′)₂ of the antibody.

8. A method according to claim 1, wherein the second beads comprise a streptavidin coating and a biotinylated F(ab′)₂ fragment having affinity for the Fc fragment.

9. A method according to claim 1, wherein, in the eluting step (d), the acidic eluent comprises about 1% formic acid and about 25% acetonitrile, volume/volume.

10. A method for analyzing whether, in an antibody-drug conjugate (ADC) having a first partner molecule attached to the Fc fragment of the antibody and a second partner molecule attached to the Fd′ fragment of the antibody, a transformation has occurred in either the first or second partner molecule, comprising the steps of:
a. capturing the ADC on first beads having an affinity for the F(ab′)₂ region of the antibody;
b. digesting the ADC captured on the first beads in a digestion medium comprising a protease that cleaves the antibody into an F(ab′)₂ fragment having the second partner molecule attached thereto and an Fc fragment having the first partner molecule attached thereto and optionally a deglycosylating enzyme, whereby the F(ab′)₂ fragment remains attached to the first beads and the Fc fragment is released;
c. capturing the released Fc fragment having the first partner molecule attached thereto on beads having an affinity for the Fc fragment;
d. eluting with an acidic eluent the Fc fragment having the first partner molecule attached thereto from the second beads;
e. eluting with an acidic solvent the F(ab′)₂ fragment having the second partner molecule attached thereto from the first beads;
f. reducing disulfide bonds in the eluted F(ab′)₂ to produce an Fd′ fragment having the second partner molecule attached thereto;
and
e. obtaining a mass spectrometric analysis of the eluted Fc fragment containing the first partner molecule attached thereto and the Fd′ fragment having the second partner molecule attached thereto, to determine if a transformation occurred in the first and second partner molecules.

11. A method according to claim 10, wherein the antibody in the ADC is a human antibody.

12. A method according to claim 11, wherein the protease is IdeS.

13. A method according to claim 11, wherein the antibody is glycosylated.

14. A method according to claim 13, wherein the digestion medium comprises a deglycosylating enzyme selected from the group consisting of PNGase and EndoS2.

15. A method according to claim 11, wherein the antibody lacks glycan groups.

16. A method according to claim 10, wherein the first beads comprise a streptavidin coating and a biotinylated F(ab′)₂ fragment having affinity for the F(ab′)₂ of the antibody.

17. A method according to claim 10, wherein the second beads comprise a streptavidin coating and a biotinylated F(ab′)₂ fragment having affinity for the Fc fragment.

18. A method according to claim 10, wherein, in the eluting steps (d) and (e), the acidic eluent comprises about 1% formic acid and about 25% acetonitrile, volume/volume.

19. A method according to claim 10, wherein the first and second partner molecules are the same.

20. A method according to claim 10, wherein the first and second partner molecules are different.

21. A method according to claim 10, wherein the antibody further has a third partner molecule attached to its light chain.
